(19) United States
(12) Patent Application Publication
(10) Pub. No.: US 2020/0368170 A1
(43) Pub. Date: Nov. 26, 2020

(54) A LIPID NANOPARTICLE COMPOSITION AND A PHARMACEUTICAL COMPOSITION FOR TREATING A HEMATOPROLIFERATIVE DISORDER

(71) Applicant: Jiangsu Genalza Biotech Co., Ltd., Nantong (CN)

(72) Inventors: Hao Feng, Shanghai (CN); Yibai Li, Shanghai (CN)

(73) Assignee: Jiangsu Genalza Biotech Co., Ltd., Nantong (CN)

(21) Appl. No.: 16/635,858

(22) PCT Filed: Jul. 31, 2018
(86) PCT No.: PCT/CN2018/097770

§ 371 Date: Jan. 31, 2020

(30) Foreign Application Priority Data
Jul. 31, 2017 (CN) PCT/CN2017/095241

(51) Int. Cl. A61K 9/50; A61K 31/7068; A61K 31/704; A61K 9/19; A61P 35/02

(52) U.S. Cl. CPC A61K 9/50; A61K 31/7068; A61P 35/02; A61K 9/19; A61K 31/704

(57) ABSTRACT

The present invention provides a lipid nanoparticle composition and a pharmaceutical composition thereof used in treating a hematoproliferative disorder. Specifically, the lipid nanoparticle composition may include cytarabine, an anthracycline, and lipid nanoparticles, wherein cytarabine and the anthracycline are co-encapsulated in the lipid nanoparticles, the lipid nanoparticles comprise a charged lipid stabilizer, and the effective mean particle size of the lipid nanoparticles is less than 400 nm.

### TECHNICAL FIELD

The present invention relates to the field of pharmaceutical formulation, more particularly relates to a lipid nanoparticle composition and a pharmaceutical composition thereof for treating a hematoproliferative disorder.

### BACKGROUND

Hematoproliferative disorders comprises leukemia, malignant lymphoma, multiple myeloma and the like. Acute myeloid leukemia (AML) is a common type of hematoproliferative disorder. The “7+3” therapy: an acronym for a combination of standard dosage of cytarabine with daunorubicin or 4-demethoxydaunorubicin, is a standard treatment for AML endorsed by international guidelines. Cytarabine (Ara-C) is a phase-specific antitumor agent, which exerts significant inhibitory effects on cell proliferation during S phase of cell division. In cells, cytarabine is converted to its active metabolite, cytarabine-5′-triphosphate (Ara-CTP). Ara-CTP has been demonstrated to be an inhibitor of DNA polymerase. Cytarabine produces cytotoxic effects in a variety of mammalian cell cultures in vitro.

4-Demethoxydaunorubicin is an anthracycline that primarily used as a therapeutic agent for hematoproliferative disorders in clinical settings. In cells, 4-Demethoxydaunorubicin intercalates itself into DNA base pairs, preventing extension, replication, and transcription of DNA strands, which eventually leads to cell apoptosis. It has recently been shown that 4-demethoxydaunorubicin may further affect activities of topoisomerase II (Top II), an enzyme that plays an important role in maintenance of normal spatial structure of DNA and ensuring process of DNA replication and transcription. Annamycin is a new generation anthracycline, which is associated with less severe cardiotoxicity compared with other anthracyclines. Annamycin has been used as a monotherapy for acute myeloid leukemia in clinical studies.

Conventional “7+3” combination therapy has a limited efficacy benefit. In standard dosing regimen, one or more agents may undergo rapid clearance before reaching the target disease site. In the case of concomitant administration, rapid clearance of one agent but not the other may lead to a failure of maintenance of desired ratio of two components, which can cause reduction of efficacy and increased safety risk of the combination. Meanwhile, one course of 7+3 therapy consists of 7 days (1 hour per day) of consecutive cytarabine administration. This regimen is not just associated with long hospitalization time and high expense, but may also expose patients to an increased risk of complications.

Despite its limitation, anthracyclines and cytarabine remain to be the standard agents in the induction therapy of acute myeloid leukemia during the past 30 years. There is an urgent demand of new pharmaceutical compositions that can significantly improve overall survival of patients with hematoproliferative disorders.

### SUMMARY

Provided are a lipid nanoparticle composition and a pharmaceutical composition for treating a hematoproliferative disorder. The lipid nanoparticle composition of the present invention and the pharmaceutical composition thereof can significantly improve overall survival of patients.

In one aspect, provided is a lipid nanoparticle composition, the lipid nanoparticle composition consists of cytarabine, an anthracycline and lipid nanoparticles, wherein,

cytarabine, and the anthracycline are co-encapsulated in the lipid nanoparticles,

the lipid nanoparticles comprise a charged lipid stabilizer, and

the effective mean particle size of the lipid nanoparticles is less than 400 nm.

In one embodiment, the anthracycline is annamycin, 4-demethoxydaunorubicin or a combination thereof. In one embodiment, the anthracycline is annamycin and/or 4-demethoxydaunorubicin.

In one embodiment, cytarabine further comprises a pharmaceutically acceptable salt thereof.

In one embodiment, the anthracycline further comprises a pharmaceutically acceptable salt thereof.

In one embodiment, the molar ratio of cytarabine to the anthracycline is from 2:1 to 50:1.

In one embodiment, the components of lipid nanoparticles comprise at least one phosphatidylcholine, a charged lipid stabilizer and a conditioning agent of phospholipid membrane fluidity.

In one embodiment, the lipid nanoparticles are in liquid form or lyophilized form.

In another aspect, further provided is a pharmaceutical composition, which comprises the lipid nanoparticle composition of the present invention and a pharmaceutically acceptable carrier.

In yet another embodiment, the pharmaceutical composition is in liquid form or lyophilized form.

In one or more embodiments, the use of the lipid nanoparticle composition or the pharmaceutical composition is provided in the preparation of a medicament for treating a hematoproliferative disorder.

In another aspect, provided is a method of treating a hematoproliferative disorder, the method comprises administrating an effective amount of the lipid nanoparticle composition or the pharmaceutical composition of the present invention to a subject in need thereof.

In yet another aspect, provided is a lipid nanoparticle composition or a pharmaceutical composition, for use in treatment of a hematoproliferative disorder.

In one embodiment, the hematoproliferative disorder is leukemia, malignant lymphoma or multiple myeloma.

### BRIEF DESCRIPTION OF THE DRAWINGS

FIG. 1. Effect of the lipid nanoparticle composition (cytarabine dosage 12 mg/kg) of example 3, example 5, comparative example 1 and comparative example 2 on leukemia-bearing DBA/2J mice.

FIG. 2. Effect of the lipid nanoparticle composition (cytarabine dosage 12 mg/kg) of example 3, example 4 and comparative example 3 and the lipid nanoparticle composition (cytarabine dosage 15 mg/kg) of example 3 and example 4 on leukemia-bearing DBA/2J mice.

### DETAILED DESCRIPTION

The technical content of the present invention is described below through specific embodiments, and those skilled in the art can easily understand other advantages and effects of the present invention from the disclosure of the present specification. The invention may also be embodied or applied by other different embodiments. Various modifications and changes can be made by those skilled in the art without departing from the scope of the invention.

### Definitions

Unless defined otherwise, all technical and scientific terms used herein have the same meaning. Otherwise, the terms and phrases used herein have the meanings listed below. When a trade name appears in this document, it is intended to refer to its corresponding commodity or its active ingredient. All patents, published patent applications and publications cited herein are hereby incorporated by reference.

The terms “about” and “approximately” when used in conjunction with a numerical variable, generally mean that the value of that variable and all values of that variable are within experimental error (for example, within a 95% confidence interval for the mean) or ±10 of the specified value, or a wider range.

Those skilled in the art will understand that when describing the components of a pharmaceutical composition, their respective percentages are based on the total weight of the pharmaceutical composition. It should be understood that when the content of the components in the composition is described as a percentage, the sum of percentages of all components is 100%.

The ranges (such as ranges of values) listed herein may encompass each value in the range and each sub-range formed by each value. For example, the expression “the molar ratio of cytarabine to the anthracycline is from 30:1 to 50:1” encompasses every point value and sub-range from 30:1 to 50:1, such as 30:1-35:1, 30:1-40:1, 30:1-45:1, and it may be an integer or a decimal, such as 30:1, 31:1, 32:1, 33:1, 34:1, 59:2, 61:2, 63:2, 100:3, and the like.

Unless otherwise stated herein, the singular forms, for example “a” and “the”, comprises plural forms. The expression “one or more” or “at least one” may indicate 1, 2, 3, 4, 5, 6, 7, 8, 9 or more.

The term “Pharmaceutically acceptable salt” used herein refers to an organic salt and an inorganic salt of the compound of the invention. The pharmaceutically acceptable salt is known to a person skilled in the art, like those recited in the literature: S. M. Berge et al., J. Pharmaceutical Sciences, 66: 1-19, 1977. Pharmaceutically acceptable salts formed by non-toxic acids include, but not limited to, a salt formed by reaction with an inorganic acid, e.g. hydrochloride, hydrobromate, phosphate, sulfate, perchlorate; and a salt formed by reaction with an organic acid, e.g, acetate, oxalate, maleate, tartrate, citrate, succinate, malonate, or a salt obtained through other methods recited in the literatures, like ion exchange. Other pharmaceutically acceptable salts include but are not limited to adipate, alginate, ascorbate, aspartate, benzenesulphonate, benzoate, bisulphate, borate, butyrate, camphorate, camphorsulfonate, cyclopentanepropionate, digluconate, dodecyl sulfates, ethanesulfonate, formate, fumarate, glucoheptonate, glycerin phosphate, glyconate, hemisulphate, heptylate, hexanoate, hydriodate, 2-hydroxy-ethanesulfonate, lactobionate, lactate, laurate, lauryl sulfate, malate, malonate, methanesulfonate, 2-naphthalenesulfonate, nicotinate, nitrate, oleate, palmitate, pamoate, pectate, persulphate, 3-phenylpropionate, picrate, pivalate, propionate, stearate, thiocyanate, p-methyl benzenesulphonate, undecylate, valerate, etc. Salts obtained from suitable bases include but are not limited to salts of alkali metal, alkali-earth metal, ammonium and N+(C₁₋₄ alkyl)₄. Quaternary ammonium salt formed by any nitrogen-containing compound is also encompassed by the invention. Water-soluble or oil-soluble or dispersion product may be obtained by quaternization. The alkali metal or alkali-earth metal salts include sodium, lithium, potassium, calcium, magnesium salt, etc.

“Anthracycline” is a type of antitumor antibiotic with an anthracene ring in its structure. Some anthracyclines that has an anthraquinone structure are also called “anthraquinone antibiotic”. Examples of anthracyclines include, but not limited to, doxorubicin, daunorubicin, 4-demethoxydaunorubicin, epirubicin, zorubicin, aclarubicin, mitoxantrone, bisantrene, annamycin and the like.

The term “lipid” refers to organic compound with property of lipophilicity or amphiphilicity. Examples thereof include, but not limited to, fat, fat oil, essential oil, waxes, steroids, sterols, phospholipids, glycolipids, sulfonyllipids, aminolipids, lipochromes and fatty acids. The term “lipid” includes both natural and synthetic lipids.

The term “liposome” refers to a type of vesicle that is generally composed of a lipid, particularly a phospholipid. A liposome generally has an aqueous/hydrophilic cavity that may be used for encapsulation of active compounds. Encapsulation of the medicament in liposomes of the invention may be carried out using any method known in the art. For a review of methods for liposome preparation and application, see, for example, Gregoriadis G, ed. Liposome Technology, 3rd ed. London: Informa Healthcare, 2006. Generally speaking, the surface charge of a liposome is determined by the positive and/or negative charges and the combination thereof carried by its components (such as phospholipids) at a certain pH. Those skilled in the art may understand that, the surface charge of liposome may be adjusted using any method known in the art, for example, negative charges may be introduced to liposome by adding an acidic lipid, such as phosphatidic acid (RA) and phosphatidylserine (PS), for example, positive charges may be introduced to liposome by adding a base (amino) lipid, such as octadecylamine and the like. Examples of other positively charged lipids include, but not limited to, stearamide, a positively charged oleoyl fatty amine derivative (such as N-[1-(2,3-dioleoyl)propyl-]-N,N,N-trimethylammonium chloride), a positively charged cholesterol derivative (such as 3β-[N-(N′,N′-dimethylaminoethane)-carbamoyl]cholesterol hydrochloride) and the like.

As used herein, the term “lipid stabilizer” refers to an agent that has effect(s) of enhancing the physical/chemical stability of a membrane, improving its pharmacokinetic properties, and/or reducing/avoiding undesirable rapid in vivo elimination of liposomes. For example, an agent that enhances the lipid membrane stability by adjusting lipid membrane charge, such as a phospholipid or another lipid, examples of which include, but not limited to, one or more of phosphatidic acid, phosphatidylserine, phosphatidylglycerol and cholesterol sulfate. For example, modified lipids obtained by covalently binding a desired functional group on lipids, particularly phospholipids and other lipids modified by polyethylene glycol and/or substituted polyethylene glycol, such as PEGylated phospholipids, particularly methoxypolyethylene glycol-distearylphosphatidylethanolamine. “Charged lipid stabilizer” refers to the presence of charge on lipid stabilizers, such as positive charge or negative charge.

The term “a conditioning agent of phospholipid membrane fluidity” refers to a molecule capable of affecting the structure of a phospholipid membrane, particularly the arrangement of aliphatic chains of the molecules that composes phospholipid membrane and thereby changing the fluidity of the membrane, for example cholesterol.

The term “encapsulating” or synonymous “incorporating”, “embedding” and the like, or “encapsulating” a component within a lipid, refers to the encapsulation of a specific component into a vesicle which is composed of a lipid bilayer.

As used herein, the “effective mean particle size” refers to volume weighted mean particle size. It may be measured by dynamic light scattering method, for example, by 380ZLS nanometer particle size and potential analyzer of PSS Company of the United States.

The expression “therapeutic advantage” refers to improved therapeutic effect or associated with less serious or lower incidence rate of adverse reaction. When a particular active substance or the pharmaceutical composition is described to have therapeutic advantage over other treatment agents, including but not limited to, when used alone or in combination with other treatment agent/treatment method, it exhibits greater in vivo/in vitro potency/efficacy, improvement of clinical performance (such as improvement observed for any hematologic or non-hematologic indicator relevant associated with treatment, or longer survival achieved, etc.), has better pharmacokinetic parameters (such as half-life) which leads to stronger therapeutic effect and/or lower toxicity, and the like. When a particular pharmaceutical composition is described to have pharmacodynamic advantage, including but not limited to, its components have greater in vivo/in vitro activity and/or pharmacokinetic profile, for example, in vivo release and distribution and half-life of one or more components enables multi-components to exert therapeutic effect together and/or reduce or avoid adverse reaction.

The term “pharmaceutically acceptable carrier” refers to the carrier substances that have no notable irritation to the organism and do not derogate the bioactivities or performance of the active compound. “Pharmaceutically acceptable carrier” includes, but not limited to, glidants, sweetening agents, diluting agents, preservatives, dyes/coloring agents flavoring agents, surfactants, wetting agents, dispersants, disintegrate suspending agents, stabilizers, isotonic agents, solvents or emulsifiers. Non-limiting examples of the carriers include calcium carbonate, calcium phosphate, various saccharides and various starch, cellulose derivatives, gelatin, vegetable oil, polyethylene glycol, and the like. For additional information about carriers, see Remington: The Science and Practice of Pharmacy, 21st Ed., Lippincott, Williams & Wilkins (2005), of which the contents are incorporated herein by reference.

The term “administering” or “administration” and the like refers to a method by which a compound or composition can be delivered to a desired biological site of action. These methods include, but not limited to, parenteral (including intravenous, subcutaneous, intraperitoneal, intramuscular, intravascular injection or infusion), topical, rectal administration, and the like.

The term “treatment” includes abating, alleviating or ameliorating a disease or symptom, preventing additional symptoms, ameliorating or preventing a potential metabolic factors of a symptom, inhibiting a disease or symptom, for example, arresting the regression of a disease or symptom, ameliorating a disease or symptom, promoting alleviation of a disease or symptom, or stop the condition of a disease or symptom, intended to include prophylaxis. “Treatment” further includes achieving therapeutic benefits and/or prophylactic benefits. Therapeutic benefit refers to eradication or amelioration of the condition being treated. In addition, therapeutic benefit is achieved by eradicating or ameliorating one or more physiological condition associated with the underlying disease, notwithstanding the patient may still be afflicted with the underlying disease, and an improvement in the patient's disease may be observed. Prophylactic benefit refers to that the use of the compositions may be administered to a patient to prevent the risk of a disease, or may be administered when a patient has one or more physiological condition, although the disease has not been diagnosed.

For medicament or active ingredient, the term “effective amount”, “therapeutically effective amount” or “prophylactically effectively amount” refers to a sufficient amount of medicament of medicine that shows acceptable side effects while achieving desired effect. For the injectable form of the present invention, the “effective amount” of an active substance in the composition may be the amount required to elicit the desired effect when used in combination with another active substance in the composition. The determination of effective amount varies from person to person, depending on the age and general condition of the object, also depending on the particular active substance, and the suitable effective amount in a particular case may he determined by those skilled in the art based on conventional experimentation.

The term “active ingredient”, “treatment agent”, “active substance” or “active agent” refers to a chemical entity that may effectively treat or prevent a target disorder, disease or condition.

“Composition” includes: a product comprising particular amounts of particular ingredients, and any product that is directly or indirectly formed by combination of particular amounts of particular ingredients. A pharmaceutical composition may comprise: the active ingredient and the inert ingredients as carrier, a product formed, directly or indirectly, by combining, compounding or agglomerating two or more ingredients, or a product formed by breakdown of one or more ingredients, or a product formed by another type of reaction or interaction of one or more ingredients.

The term “patient” or “subject” as used herein, refers to human (including adults and children) or other animals (including but not limited to mammals and rodents). According to some embodiments of the invention, “patient” or “subject” refers to human.

### Lipid Nanoparticle Composition

In one aspect, provided is a lipid nanoparticle composition, wherein the lipid nanoparticle composition consists of cytarabine, an anthracycline and lipid nanoparticles, wherein cytarabine and the anthracycline: are co-encapsulated in the lipid nanoparticles. The lipid nanoparticles comprise a charged lipid stabilizer, and the effective mean particle size of lipid nanoparticles is less than 400 nm.

The component-encapsulating nanostructured lipid carrier refers to the lipid nanoparticle formed by encapsulating two or more drug components into a closed vesicle with a structure that is composed of a phospholipid bilayer and similar to a cell. Components which constitute the lipid carrier include phosphatidylcholine, phosphatidylglycerol, cholesterol and the like. These lipid components are non-toxic, non-immunogenic, and with good biocompatibility. The lipid nanoparticles can be used to regulate the release rate of encapsulated medicament, achieving its sustained release. Meanwhile it can also enhance the efficacy of the medicament. The lipid nanoparticles can also protect the encapsulating drug components from enzymolysis. The nanostructured lipid carrier can encapsulate not only water-soluble medicaments (in the inner aqueous phase), but also lipid-soluble medicaments (in the bilayer).

In another embodiment, the anthracycline is annamycin and/or 4-demethoxydaunorubicin.

In one embodiment, cytarabine further comprises a pharmaceutically acceptable salt thereof.

In one embodiment, the anthracycline further comprises a pharmaceutically acceptable salt thereof.

In one embodiment, the molar ratio of cytarabine to the anthracycline is from 2:1 to 50:1. In one embodiment, the molar ratio of cytarabine to the anthracycline is from 5:1 to 40:1. In one embodiment, the molar ratio of cytarabine to the anthracycline is from 10:1 to 30:1.

In a preferred embodiment, the molar ratio of cytarabine to the anthracycline is from 30:1 to 50:1. In a preferred embodiment, the molar ratio of cytarabine to the anthracycline is from 30:1 to 40:1. In a particularly preferred embodiment, the molar ratio of cytarabine to the anthracycline is 30:1.

The lipid nanoparticle composition that encapsulates cytarabine and the anthracycline (such as annamycin and/or 4-demethoxydaunorubicin) in specific ratio can exhibit excellent efficacy. Particularly, when the molar ratio of the two components is about from 30:1 to 50:1, the survival of the leukemia model mice can be significantly improved. Meanwhile, more significant advantages over other ratios (such as about 5:1, about 15:1, about 18:1) can be exhibited.

In one embodiment, the effective mean particle size of lipid nanoparticles is less than 200 nm, such as 100 nm or less.

In one embodiment, the components of lipid nanoparticles comprise at least one phosphatidylcholine, a charged lipid stabilizer and a conditioning agent of phospholipid membrane fluidity.

In one embodiment, phosphatidylcholine is any one or more selected from egg yolk phosphatidylcholine (EPC), hydrogenated soybean phosphatidylcholine (HSPC), distearoylphosphatidylcholine (DSPC), dipalmitoylphosphatidylcholine (DPPC), dioleylphosphatidylcholine (DOPC), dimyristoylphosphatidylcholine (DMPC), preferably hydrogenated soybean phosphatidylcholine and/or distearoylphosphatidylcholine.

In one embodiment, the charged lipid stabilizer is selected from the group consisting of methoxypolyethylene glycol-distearylphosphatidylethanolamine, phosphatidylglycerol or cholesteryl sulfate. In another embodiment, the phosphatidylglycerol is selected from any one of the following or a mixture of several of the following: dimyristoylphosphatidylglycerol (DMPG), dipalmitoylphosphatidylglycerol (DPPG), dioleoylphosphatidylglycerol (DOPG), distearylphosphatidylglycerol (DSPG), preferably methoxypolyethylene glycol-distearylphosphatidylethanolamine and/or distearoylphosphatidylglycerol, or methoxypolyethylene glycol-distearylphosphatidylethanolamine and/or cholesteryl sulfate.

In one embodiment, the charged lipid stabilizer is selected from the group consisting of methoxypolyethylene glycol-distearylphosphatidylethanolamine and phosphatidylglycerol. In another embodiment, the charged lipid stabilizer is selected from the group consisting of methoxypolyethylene glycol-distearoylphosphatidylethanolamine and cholesteryl sulfate.

In one embodiment, the conditioning agent of phospholipid membrane fluidity is selected from cholesterol.

In another embodiment, the lipid nanoparticles are in liquid form or lyophilized form.

### Pharmaceutical Composition, Formulation and Kit

In another aspect, further provided is a pharmaceutical composition which comprises the lipid nanoparticle composition of the present invention and a pharmaceutically acceptable carrier.

In on embodiment, provided is a pharmaceutical composition, the pharmaceutical composition comprises the lipid nanoparticle composition of the present invention and a pharmaceutically acceptable carrier. The lipid nanoparticle composition comprises 1%˜7% by weight of cytarabine, 0.1%˜3% by weight of anthracycline, the carrier comprises 5%˜20% by weight of hydrogenated soybean phosphatidylcholine or distearoylphosphatidylcholine, 0.5%˜5% by weight of methoxypolyethylene glycol-distearylphosphatidylethanolamine, 0.5˜5% by weight of cholesterol and 70%˜90% by weight of sucrose.

Wherein, the lipid nanoparticle composition comprises 2%˜5% by weight of cytarabine, 0.1%˜1.5% by weight of the anthracycline, and the carrier comprises 6%˜1.2% by weight of hydrogenated soybean phosphatidylcholine or distearoylphosphatidylcholine, 1%˜3% by weight of methoxypolyethylene glycol-distearylphosphatidylethanolamine, 1%˜3% by weight of cholesterol and 75%˜85% by weight of sucrose.

In a specific embodiment, provided is a pharmaceutical composition, which comprises the lipid nanoparticle composition of the present invention and a pharmaceutically acceptable carrier, and the pharmaceutical composition comprises 1%˜7% by weight of cytarabine, 0.1%˜3% by weight of the anthracycline, 5%˜20% by weight of hydrogenated soybean phosphatidylcholine or distearoylphosphatidylcholine, 0.5%˜5% by weight of methoxypolyethylene glycol-disteaphosphatidylethanolamine, 0.5%˜5% by weight of cholesterol and 70%˜90% by weight of sucrose.

In a preferred embodiment, provided is a pharmaceutical composition, which comprises the lipid nanoparticle, composition of the present invention and a pharmaceutically acceptable carrier, and the pharmaceutical composition comprises 2%˜5% by weight of cytarabine, 0.1%˜1.5% by weight of the anthracycline, 6%˜12% by weight of hydrogenated soybean phosphatidylcholine or distearoylphosphatidylcholine, 1%˜3% by weight of methoxypolyethylene glycol-distearylphosphatidylethanolamine, 1%˜3% by weight of cholesterol and 75%˜85% by weight of sucrose.

In one embodiment, provided is a pharmaceutical composition which comprises the lipid nanoparticle composition and a pharmaceutically acceptable carrier of the present invention, and the lipid nanoparticles comprise 1%˜7% by weight of cytarabine, 0.1%˜3% by weight of the anthracycline, 5%˜20% by weight of hydrogenated soybean phosphatidylcholine or distearoylphosphatidylcholine, 0.5%˜1.0% by weight of distearylphosphatidylglycerol, 0.5%˜5% by weight of cholesterol and 65%˜90% by weight of sucrose.

Wherein, the lipid nanoparticle composition comprises 2%˜5% by weight of cytarabine, 0.1%˜1.5% by weight of the anthracycline, 12%˜18% by weight of hydrogenated soybean phosphatidylcholine or distearoylphosphatidylcholine, 2%˜5% by weight of distearylphosphatidylglycerol, 0.5%˜2% by weight of cholesterol and 70%˜80% by weight of sucrose.

In a specific embodiment, provided is a pharmaceutical composition, the pharmaceutical composition comprises the lipid nanoparticle composition of the present invention and a pharmaceutically acceptable carrier, and the pharmaceutical composition comprises 1%˜7% by weight of cytarabine, 0.1%˜3% by weight of the anthracycline, 5%˜20% by weight of hydrogenated soybean phosphatidylcholine or distearoylphosphatidylcholine, 0.5%˜10% by weight of distearylphosphatidylglycerol, 0.5%˜5% by weight of cholesterol and 65%˜90% by weight of sucrose.

In a preferred embodiment, provided is a pharmaceutical composition, which comprises the lipid nanoparticle composition of the present invention and a pharmaceutically acceptable carrier, and the pharmaceutical composition comprises 2%˜5% by weight of cytarabine, 0.1%˜1.5% by weight of the anthracycline, and the carrier comprises 12%˜18% by weight of hydrogenated soybean phosphatidylcholine or distearoylphosphatidylcholine, 2%˜5% by weight of distearylphosphatidylglycerol, 0.5%˜2% by weight of cholesterol and 70%˜80% by weight of sucrose.

In one embodiment, the effective mean particle size of lipid nanoparticles in pharmaceutical composition of the present invention is preferably less than 200 nm, such as 100 nm or less.

In another embodiment, the lipid nanoparticles in pharmaceutical composition of the present invention are in liquid form or lyophilized form.

The pharmaceutical composition of the invention may be administered through any route, as long as it can exhibit the effect of preventing, alleviating, avoiding or curing symptoms of human or animal. For example, various suitable dosage forms may be prepared according to the route of administration, particularly injections, such as injection solutions, sterile powder for injection or concentrated solutions for injection. Therefore, in another aspect, further provided is a pharmaceutical formulation, which comprises pharmaceutical composition of the invention. When the pharmaceutical formulation is used for parenteral administration, suitable dosage forms include, but not limited to, sterile solutions, suspensions, and lyophilized products, and the like.

In a further aspect, provided is a kit, which comprises the pharmaceutical composition of the present invention. The kit may comprise a package insert thereof.

### Preparation

The lipid nanoparticle composition and pharmaceutical composition thereof of the present invention may be prepared by any method known in the art. For example, the lipid nanoparticles in liquid form may be prepared by dispersing the lipid nanoparticle composition of the present invention in a pharmaceutically acceptable liquid carrier. It should be understood that according to the present invention, the lipid nanoparticles in liquid form may be formulated prior to or during use. The lipid nanoparticle composition of the present invention may be prepared as lyophilized formulation. The lyophilized formulation may further comprise a lyoprotectant. The lyoprotectant may be selected from the group consisting of scrose, trehalose or mannitol, preferably sucrose.

In an exemplary embodiment, the lipid nanoparticles of the present invention may he prepared by the following preparation process: dissolving an excessive amount of cytarabine in an appropriate amount of ammonium sulfate solution; dissolving phosphatidylcholine, the charged lipid and the conditioning agent of lipid membrane fluidity in an appropriate amount of absolute ethanol or 95% ethanol; mixing the two well, and then mixing by mechanical stirring at 100-500 rpm to obtain the raw lipid particles; passing the raw lipid particles through high pressure homogenizer for several cycles while keeping the material at a temperature of at least 50-70° C., extruding through a polycarbonate membrane for several times under high pressure, reducing the mean particle size to less than 200 nm; removing the unencapsulated medicament by ultrafiltration, and replacing the buffer solution with sucrose solution, adjusting the pH to 6.0-7.4, adding the anthracycline aqueous solution, keeping the material at a temperature of at least 50-70° C., keeping it for more than 20 minutes. After sub-packaging, lyophilization is conducted to obtain the lipid nanoparticles of cytarabine/anthracycline for injection.

### Treatment and Use

The lipid nanoparticle composition and the pharmaceutical composition thereof of the present invention can be used in hematoproliferative disorders. In on embodiment, the hematoproliferative disorder is leukemia (for example, acute leukemia), malignant lymphoma or multiple myeloma. In one embodiment, the hematoproliferative disorder is myeloid leukemia (for example, acute myeloid leukemia), lymphocytic leukemia, granulocytic leukemia, monoblastic leukemia, monocytic leukemia or T-cell leukemia. In one embodiment, the hematoproliferative disorder is myelodysplastic syndrome. In one embodiment, the hematoproliferative disorder is newly diagnosed, or relapsed or refractory. The lipid nanoparticle composition of the present invention and the pharmaceutical composition thereof can be administered to a subject in need thereof through any suitable route including, but not limited to, injection, transmucosal, inhalation, ocular, topical administration and the like, particularly injection, including, for example, intravenous, subcutaneous, intraperitoneal, intramuscular, intravascular injection, and the like.

The dosing regimen can be adjusted to provide the optimal desired response. For example, when administered via injection, administration may be conducted by single bolus, group bolus and/or continuous infusion and the like. For example, several partial doses may be administered over time, or may be proportionally reduced or increased as indicated by the urgent need for treatment. It is noted that the doses may vary with the type and severity of the condition to be alleviated, and may include single or multiple doses. Generally, the dosage of the treatment and the frequency of administration may vary, depending on the factors to he considered, such as age, sex, and general health of the patient to be treated; the frequency of treatment and the nature of the desired effect; the damage degree of tissue; duration of symptoms; and other variables that can be adjusted by each clinician. It is further to be understood that, for any particular subject, the specific dosing regimen will be adjusted over time according to the need of the subject and the professional judgment of the person conducting or supervising the administration of the composition. Desired result can be achieved by administering required dose for one or more times. The pharmaceutical composition according to the invention can also be provided in unit dosage form.

The lipid nanoparticle composition or the pharmaceutical composition or the corresponding treatment method can be used in combination with additional treatment methods including, but not limited to, radiotherapy, chemotherapy, immunotherapy or a combination thereof. For example, the lipid nanoparticle composition or the pharmaceutical composition of the present invention can be used as radiosensitizer to enhance the efficacy of radiotherapy. Examples of radiotherapy include external radiotherapy, teletherapy, brachytherapy, sealed source radiotherapy and open source radiotherapy and the like. For example, the lipid nanoparticle composition or the pharmaceutical composition of the present invention can be used in combination with a chemotherapeutic agent, the chemotherapeutic agent includes those known in the art. For example, the lipid nanoparticle composition or the composition of the invention can be used in combination with immunological formulation including interferon and other immunoenhancers, immunotherapeutic medicament, and the like.

### Beneficial Effect

The lipid nanoparticle composition encapsulating cytarabine and an anthracycline In the ratios according to the present invention have unexpected pharmacodynamic advantage. The lipid nanoparticles can control the release rate of the encapsulated medicament, and maintain the proportion of the encapsulated medicament for a certain period of time, meanwhile providing protection for the encapsulated medicament and improving the efficacy of the medicament. Moreover, the results of animal experiments further indicate that the lipid nanoparticle composition of the present invention can significantly improve the survival of leukemia model mice and exhibit excellent effects.

### EXAMPLES

Unless otherwise stated, the instruments and reagents used in the examples are commercially available. The reagents can be used directly without further purification.

Cytarabine and sucrose were obtained from Sigma-Aldrich company; 4-demethoxydaunorubicin was obtained from Selleck company; distearoylphosphatidylcholine, distearylphosphatidylglycerol, hydrogenated soybean phosphatidylcholine, methoxypolyethylene glycol-distearylphosphatidylethanolamine were obtained from Lipoid company; ammonium sulfate, copper gluconate, triethanolamine were obtained from Sinopharm Chemical Reagent Co., Ltd.; cholesterol was obtained from Nippon Fine Chemical company; polycarbonate membrane was obtained from Whatman company; annamycin was prepared according to the method disclosed in U.S. Pat. No. 5,977,327A.

### Example 1

### Cytarabine/Annamycin Lipid Nanoparticle Injection

Ingredients:

| | | |
|---|---|---|
| cytarabine | 50 | g |
| annamycin | 2 | g |
| distearoylphosphatidylcholine | 220 | g |
| distearylphosphatidylglycerol | 60 | g |
| cholesterol | 20 | g |
| sucrose | 1.26 | kg |
| water for injection | 10 | L |

Preparation Process:

(1) An excessive amount of cytarabine was dissolved in 10 L ammonium sulfate solution;

(2) Distearoylphosphatidylcholine, distearylphosphatidylglycerol, cholesterol and annamycin were dissolved in anhydrous ethanol at 60° C.;

(3) Ethanol in (2) was removed by rotary evaporation, then (1) was added and hydration was conducted at 60° C. for 1 h to form raw lipid particles;

(4) The raw lipid particles were passed through a high pressure homogenizer at 700 bar for 3 cycles, then extruded through 100 nm polycarbonate membrane under high pressure for once while keeping the material temperature at 60° C., and mean particle size was reduced to about 100 nm:

(5) The unencapsulated medicament was removed by ultrafiltration, and the buffer was replaced with sucrose solution;

(6) The volume of the lipid nanoparticles was adjusted to 10 L;

(7) Fill 5 mL in each of 5 mL neutral borosilicate glass vials, and add rubber stopper and cap to obtain the cytarabine/annamycin lipid nanoparticle injection.

### Example 2

### Cytarabine/4-demethoxydaunorubicin Lipid Nanoparticle Injection Ingredients:

| | | |
|---|---|---|
| cytarabine | 50 | g |
| 4-demethoxydaunorubicin (calculated in free base) | 3.5 | g |
| hydrogenated soybean phosphatidylcholine | 100 | g |
| methoxypolyethylene | 30 | g |
| glycol-distearylphosphatidylethanolamine | | |
| cholesterol | 30 | g |
| sucrose | 1 | kg |
| water for injection | 10 | L |

Preparation Process:

(1) An excessive amount of cytarabine was dissolved in 4 L ammonium sulfate solution;

(2) Hydrogenated soybean phosphatidylcholine, methoxypolyethylene glycol-distearylphosphatidylethanolamine and cholesterol were dissolved in 601 L anhydrous ethanol at 60° C.;

(3) After mixing (2) and (1), the mixture was sheared by IKA high speed shearing machine at 10,000 rpm, 6 L ammonium sulfate solution was added, then mixed at 6000 rpm. Or, after mixing (2) and (1) and the mixture was stirred by mechanical stirring at 300 rpm for 1 hour;

(4) The suspension of raw lipid particles was passed through a high pressure homogenizer at 700 bar for 2 cycles, then extruded through 100 inn polycarbonate membrane under high pressure for 3 times while keeping the material temperature at about 60° C., so that the mean particle size was reduced to about 100 nm;

(5) The unencapsulated medicament was removed by ultrafiltration. Concentration was conducted, resulting in 7 L. The pH was adjusted to 6.50. 4-demethoxydaunorubicin sucrose aqueous solution was added. The temperature was kept at 60° C. for 30 minutes.

(6) The volume of the final lipid nanoparticles was adjusted to 10 L;

(7) Fill 5 mL in each of 5 mL neutral borosilicate glass vials, and add rubber stopper and cap to obtain cytarabine/4-demethoxydaunorubicin lipid nanoparticle injection. The molar ratio of cytarabine to 4-demethoxydaunorubicin in the cytarabine/4-demethoxydaunorubicin lipid nanoparticles was about 30:1.

### Example 3

### Cytarabine/4-demethoxydaunorubicin Lipid Nanoparticle Injection

Ingredients:

| | | |
|---|---|---|
| cytarabine | 50 | g |
| 4-demethoxydaunorubicin (calculated in free base) | 3.5 | g |
| distearoylphosphatidylcholine | 230 | g |
| distearylphosphatidylglycerol | 70 | g |
| cholesterol | 15 | g |
| sucrose | 1.26 | kg |
| water for injection | 10 | L |

Preparation Process:

(1) An excessive amount of cytarabine was dissolved in 10 L ammonium sulfate solution;

(2) Distearoylphosphatidylcholine, distearylphosphatidylglycerol and annamycin were dissolved in anhydrous ethanol at 60° C.;

(3) The two were mixed well, and then sheared by IKA high speed shearing machine at 9000 rpm. Or, the two were mixed well and then stirred by mechanical stirring at 300 rpm for 1 hour;

(4) The suspension of raw lipid particles was passed through a microfluidic homogenizer at 2000 bar for 1 cycle, then extruded through 100 nm polycarbonate membrane under high pressure for 3 times while keeping the material temperature at about 60° C., so that the mean particle size was reduced to about 100 nm;

(5) The unencapsulated medicament was removed by ultrafiltration. The buffer was replaced with sucrose buffer solution. Concentration was conducted, resulting in 7 L. The pH was adjusted to 6.50. 4-demethoxydaunorubicin sucrose aqueous solution was added. The temperature was kept at 60° C. for 30 minutes;

(6) The volume of the final lipid nanoparticles was adjusted to 10 L;

(7) Fill 5 mL in each of 20 mL neutral borosilicate glass vials. After lyophilization, cytarabine/4-demethoxydaunorubicin lipid nanoparticle injection was obtained. The molar ratio of cytarabine to 4-demethoxydaunorubicin in the cytarabine/4-demethoxydaunorubicin lipid nanoparticles was about 30:1.

### Example 4

### Cytarabine/4-demethoxydaunorubicin Lipid Nanoparticle Injection

Ingredients:

| | | |
|---|---|---|
| cytarabine | 50 | g |
| 4-demethoxydaunorubicin (calculated in free base) | 2.56 | g |
| distearoylphosphatidylcholine | 230 | g |
| distearylphosphatidylglycerol | 70 | g |
| cholesterol | 15 | g |
| sucrose | 1.26 | kg |
| water for injection | 10 | L |

Preparation Process:

Cytarabine/4-demethoxydaunorubicin lipid nanoparticle injection was prepared according to the method of Example 3, except that the amount of 4-demethoxydaunorubicin (calculated in free base) was changed to 2.56 g. The molar ratio of cytarabine to 4-demethoxydaunorubicin in the cytarabine/4-demethoxydaunorubicin lipid nanoparticles was 40:1.

### Example 5

### Cytarabine/4-demethoxydaunorubicin Lipid Nanoparticle Injection

Ingredients:

| | | |
|---|---|---|
| cytarabine | 50 | g |
| 4-demethoxydaunorubicin (calculated in free base) | 2.1 | g |
| distearoylphosphatidylcholine | 230 | g |
| distearylphosphatidylglycerol | 70 | g |
| cholesterol | 15 | g |
| sucrose | 1.26 | kg |
| water for injection | 10 | L |

Preparation Process:

(1) An excessive amount of cytarabine was dissolved in 10 L copper gluconate triethanolamine solution;

(2) Distearoylphosphatidylcholine, distearylphosphatidylglycerol and cholesterol were dissolved in anhydrous ethanol at 60° C.;

(3) After adding (2) to (1) and mixed, continue to stir at 60° C. for 1 hour;

(4) The suspension of raw lipid particles was extruded through 200 nm and 100 nm polycarbonate membrane in sequence, each for 10 times, while keeping the material temperature at about 60° C., so that the mean particle size was reduced to about 100 nm;

(5) The unencapsulated medicament was removed by ultrafiltration, the buffer was replaced with sucrose buffer solution. Concentration was conducted, resulting in 7 L. 4-demethoxydaunorubicinsucrose aqueous solution was added, and the temperature was kept at 50° C. for 30 minutes;

(6) The volume of the final lipid nanoparticles was adjusted to 10 L;

(7) Fill 5 mL in each of 20 mL neutral borosilicate glass vials. After lyophilization, cytarabine/4-demethoxydaunorubicinlipid nanoparticle injection was obtained. The molar ratio of cytarabine to 4-demethoxydaunorubicin in the cytarabine/4-demethoxydaunorubicin lipid nanoparticles was about 50:1.

### Comparative Example 1

### Cytarabine/4-demethoxydaynorubicin Lipid Nanoparticle Injection

Ingredients:

| | | |
|---|---|---|
| cytarabine | 50 | g |
| 4-demethoxydaunorubicin (calculated in free base) | 20.45 | g |
| distearoylphosphatidylcholine | 230 | g |
| distearylphosphatidylglycerol | 70 | g |
| cholesterol | 15 | g |
| sucrose | 1.26 | kg |
| water for injection | 10 | L |

Preparation Process:

Cytarabine/4-demethoxydaunorubicin lipid nanoparticle injection was prepared according to the method of Example 5, except that the amount of 4-demethoxydaunorubicin (calculated in free base) was changed to 20.45 g. The molar ratio of cytarabine to 4-demethoxydaunorubicin in the cytarabine/4-demethoxydaunorubicin lipid nanoparticles was 5:1.

### Comparative Example 2

### Cytarabine/4-demethoxydaunorubicin Lipid Nanoparticle Injection

Ingredients:

| | | |
|---|---|---|
| cytarabine | 50 | g |
| 4-demethoxydaunorubicin (calculated in free base) | 7 | g |
| distearoylphosphatidylcholine | 230 | g |
| distearylphosphatidylglycerol | 70 | g |
| cholesterol | 15 | g |
| sucrose | 1.26 | kg |
| water for injection | 10 | L |

Preparation Process:

An excessive amount of cytarabine was dissolved in 10 L copper gluconate triethanolamine solution;

(1) Distearoylphosphatidylcholine, distearylphosphatidyldycerol and cholesterol were dissolved in anhydrous ethanol at 60° C.;

(2) was added to (1) and mixed well, then stirring was continued at 60° C. for 1 hour;

(3) The suspension of raw lipid particles was extruded through 200 inn and 100 nm polycarbonate membrane in sequence, each for 10 times, while keeping the material temperature at about 60° C., so that the mean particle size was reduced to about 100 nm;

(4) The encapsulated medicament was removed by ultrafiltration, the buffer was replaced with sucrose buffer solution. Concentration was conducted, resulting in 7 L. 4-demethoxydaunorubicinsucrose aqueous solution was added, and the temperature was kept at 50° C. for 45 minutes;

(5) The unencapsulated medicament was removed by ultrafiltration. The buffer was replaced with sucrose buffer solution and concentrated, and the volume of the final lipid nanoparticles was adjusted to 10 L. Fill in 20 mL in each of 50 mL neutral borosilicate glass vials. After lyophilization, cytarabine/4-demethoxydaunorubicin lipid nanoparticle injection was obtained. The molar ratio of cytarabine to 4-demethoxydaunorubicin in the cytarabine/4-demethoxydaunorubicin lipid nanoparticles was about 15:1.

### Comparative Example 3

### Cytarabine/4-demethoxydaunorubicin Lipid Nanoparticle Injection

Ingredients:

| | | |
|---|---|---|
| cytarabine | 50 | g |
| 4-demethoxydaunorubicin (calculated in free base) | 5.68 | g |
| distearoylphosphatidylcholine | 230 | g |
| distearylphosphatidylglycerol | 70 | g |
| cholesterol | 15 | g |
| sucrose | 1.26 | kg |
| water for injection | 10 | L |

Preparation Process:

Cytarabine/4-demethoxydaunorubicin lipid nanoparticle injection was prepared according to the method of comparative example 2, except that the amount of 4-demethoxydaunorubicin (calculated in free base) was changed to 5.68 g. The molar ratio of cytarabine to 4-demethoxydaunorubicin in the cytarabine/4-demethoxydaunorubicin lipid nanoparticles was 18:1.

### Effect Example

### Effect on the Survival of Leukemia-Bearing Mice

The lipid nanoparticles encapsulated with cytarabine/4-demethoxydaunorubicin that were prepared according to the present invention were subjected for experiments on a leukemic animal model.

Animals and materials: female DBA/2J mice (6-8 weeks) were obtained from Nanjing University-Nanjing Institute of Biomedical Research. P388D1 cells were obtained from the cell bank of the Chinese Academy of Sciences. DMED medium was purchased from Gibco company. Horse serum was purchased from BI company. Phosphate buffered saline (PBS) was obtained from Gibco company. Cell counter: Life technologies Countess II.

### Effect Example 1

Experimental Method:

P388D1 cells were cultured in DMEM+10% horse serum medium. On the first day of the experiment, P388D1 cells were collected by aspiration, and centrifuged at 125 g for 5 minutes. The supernatant was removed. Cells were then resuspended in 1 mL of sterile saline solution before were centrifuged again at 125 g for 5 minutes. After removal of supernatant, cells were then resuspended in sterile PBS solution with a count of cells adjusted to about 2.5×10⁶ cells/ml for intraperitoneal inoculation.

Each individual of 50 female DBA/2J mouse (age: 6-8 weeks) was intraperitoneally injected with 0.2 ml of P388D1 cell suspension in PBS, before randomized into 5 treatment groups, N=10/group.

On the 3^{rd}, 6^{th}, 9^{th} day after cell inoculation (which was the 4^{th}, 7^{th}, 10^{th} day of the experiment, respectively), depends on its corresponding treatment group, mice were intravenously injected with one of the following (5 ml/kg); saline (vehicle), lipid nanoparticle composition of example 3 (Lip-C₁₂&I E3), lipid nanoparticle composition of example 5 (Lip-C₁₂&I E5), lipid nanoparticle composition of comparative example 1 (Lip-C₁₂&I C1), lipid nanoparticle composition of comparative example 2 (Lip-C₁₂&I C2), wherein the dose of cytarabine in each group was 12 mg/kg.

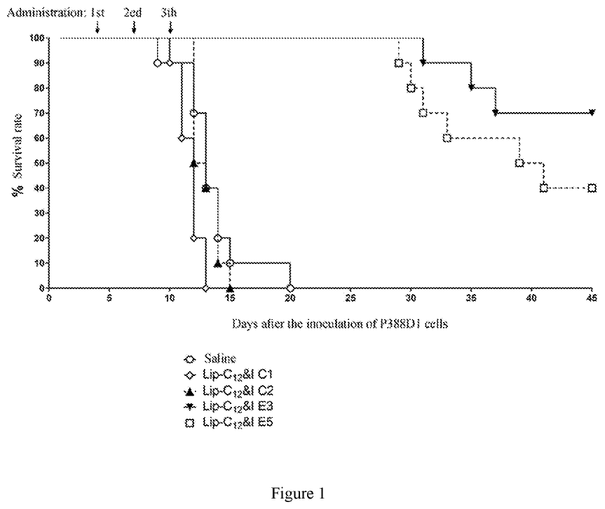

Results: Results are shown in FIG. 1. The overall survival of animal at the 45^{th} day after cell inoculation and the median survival of animal post each treatment were calculated using GraphPad Prism 5.0 software and are listed in Table 1.

TABLE 1
Overall survival at the 45^{th} day after inoculation and the
median survival of mice inoculated with P388D1 cell.

| | Overall survival rate at the 45^{th} | Median survival |
|---|---|---|
| Administration | day after inoculation | (day) |
| Saline | 0 | 12.5 |
| Lip-C₁₂&I C1 | 0 | 11 |
| (Example 1) | | |
| Lip-C₁₂&I C2 | 0 | 12 |
| (Example 2) | | |
| Lip-C₁₂&I E3 | 70% | >45 |
| (Example 3) | | |
| Lip-C₁₂&I E5 | 40% | 39.5 |
| (Example 5) | | |

Data in FIG. 1 and Table 1 indicate that, at an equal dose of cytarabine (12 mg/kg), the effect of the cytarabine:4-demethoxydaunorubicin encapsulating lipid nanoparticle composition of the present invention on DBA/2J mice inoculated with P388D1 cells was associated with the ratio of the two components it encapsulates. Therapeutic effects were observed in leukemia-bearing animals receiving lipid nanoparticle compositions with cytarabine: 4-demethoxydaunorubicin encapsulated in a molar ratio of about 30:1 (Lip-C₁₂&I E3) and 50:1 (Lip-C₁₂&I E5). Overall survival rate at the 45^{th} day after inoculation for animals receiving Lip-C₁₂&I E3 and Lip-C₁₂&I E5 were 70% and 40%, respectively. Whereas the overall survival rate of saline group animals on the 20^{th} day after inoculation was 0%. Lipid nanoparticles with cytarabine:4-demethoxydaunorubicin in a molar ratio of 5:1 (Lip-C₁₂&I C1) and about 15:1 (Lip-C₁₂&I C2) provided no therapeutic effect on model animals, and the overall survival rate at the 20^{th} day after inoculation and the median survival of the animals was not different from that of saline-treated animals.

### Effect Example 2

Experimental method: P388D1 cell suspended in PBS was prepared according to the method described in effect example 1. Female DBA/2J mice (Age: 6-8 weeks) were intraperitoneally inoculated with 0.2 ml (approximately a count of 5×10⁵) P388D1 cells before randomized into 6 groups, N=8/group.

On the 3^{rd}, 6^{th}, 9^{th} day after cell inoculation (that was the 4^{th}, 7^{th}, 10^{th} day of the experiment), mice in each group were administered with (as intravenous injection, 5 ml/kg) one of the following: saline (vehicle); the lipid nanoparticle composition of example 3, wherein the dose of cytarabine was 12 mg/kg (Lip-C₁₂&l E3); the lipid nanoparticle composition of example 3, wherein the dose of cytarabine was 15 mg/kg (Lip-C₁₅&I E3); the lipid nanoparticle composition of example 4, wherein the dose of cytarabine was 12. mg/kg (Lip-C₁₂&I E4); the lipid nanoparticle composition of example 4, wherein the dose of cytarabine was 15 mg/kg (Lip-C₁₅&I E4); the lipid nanoparticle composition of comparative example 3, wherein the dose of cytarabine was 12 mg/kg (Lip-C₁₂&I C3).

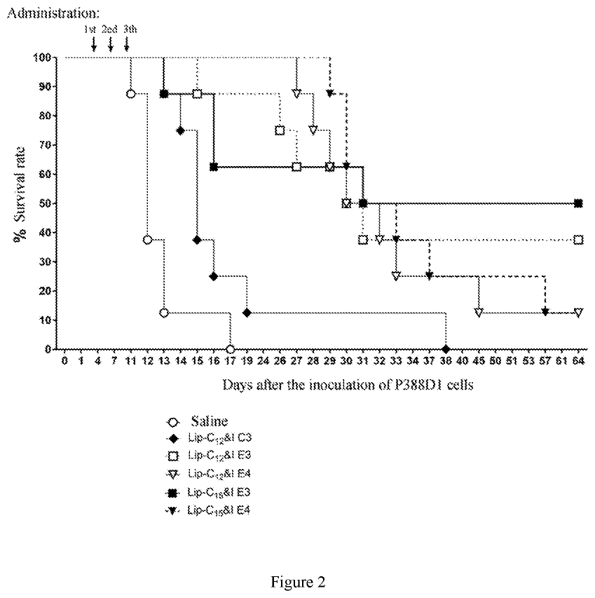

Results: Results are shown in FIG. 2. The overall survival rate at the 64^{th} day after cell inoculation and the median survival of the animals post each treatment were calculated using GraphPad Prism 5.0 software and are listed in Table 2.

TABLE 2
Overall survival at the 64^{th} day after inoculation and the
median survival of mice inoculated with P388D1 cells.

| | Overall survival rate at the | Median survival |
|---|---|---|
| Administration | 64^{th} day after inoculation | (day) |
| Saline | 0 | 9 |
| Lip-C₁₂&I C3 | 0 | 12 |
| (Comparative | | |
| example 3) | | |
| Lip-C₁₂&I E3 | 37.5% | 27.5 |
| (Example 3) | | |
| Lip-C₁₂&I E4 | 12.5% | 28 |
| (Example 4) | | |
| Lip-C₁₅&I E3 | 50% | 43 |
| (Example 3) | | |
| Lip-C₁₅&I E4 | 12.5% | 29 |
| (Example 4) | | |

Data in FIG. 2 and Table 2 indicate that, at a dose of cytarabine at 12 mg/kg, therapeutic effects were observed in mice receiving lipid nanoparticle compositions with cytarabine:4-demethoxydaunorubicin encapsulated in a molar ratio of about 30:1 (Lip-C₁₂&I E3) and 40:1 (Lip-C₁₂&I E4), with an overall survival rate of animals at the 64^{th} day after inoculation of 37.5% and 12.5%, respectively. In contrast, no therapeutic effect was observed in mice receiving a lipid nanoparticle composition with cytarabine:4-demethoxydaunorubicin in a molar ratio of about 18:1 (Lip-C₁₂&I C3), as the overall survival rate of animal at the 64^{th} day after inoculation and the median survival of animal was comparable to that of saline-treated animals. At a dose of cytarabine at 15 mg/kg, therapeutic effects were observed in mice receiving lipid nanoparticle compositions with a molar ratio of cytarabine:4-demethoxydaunorubicin of about 30:1 (Lip-C₁₅&I E3) and 40:1 (Lip-C₁₅&I E4), with an overall survival rate of animals at the 64^{th} day after inoculation of 50% and 12.5%, respectively. Among the groups, example 3 that was the lipid nanoparticle composition with a molar ratio of cytarabine:4-demethoxydaunorubicin of about 30:1, at a dose of cytarabine at 15 mg/kg (Lip-C₁₅&I E3) showed the greatest efficacy.

Taken together from the data in effect example 1 and effect example 2, at a molar ratio of cytarabine:4-demethoxydaunorubicin at about 30:1-50:1, cytarabine and 4-demethoxydaunorubicin encapsulating liposome produced therapeutic effects on the experimental animals. On the contrary, at a molar ratio of the two compounds at 5:1 to 18:1, no therapeutic effect was observed on the experimental animals.

The embodiments above are only an illustrative description of the principle, application and effect of the invention, and are not intended to limit the invention. Modifications and variations to the examples can be made by those skilled in the art without departing from the spirit and scope of the invention. Moreover, without contradiction to each other, conjunctions and combinations of various examples and various features from the examples in the present description can be employed by those skilled in the art without departing from the spirit and scope of the invention.

1. A lipid nanoparticle composition, comprising:
cytarabine, an anthracycline, and lipid nanoparticles, wherein
cytarabine and the anthracycline are co-encapsulated in the lipid nanoparticles,
the lipid nanoparticles comprise a charged lipid stabilizer, and
the effective mean particle size of the lipid nanoparticles is less than 400 nm.

2. The lipid nanoparticle composition of claim 1, wherein the anthracycline is annamycin and/or 4-demethoxydaunorubicin.

3. The lipid nanoparticle composition of claim 1, wherein cytarabine further comprises a pharmaceutically acceptable salt thereof.

4. The lipid nanoparticle composition of claim 1, wherein the anthracycline further comprises a pharmaceutically acceptable salt thereof.

5. The lipid nanoparticle composition of claim 1, wherein the molar ratio of cytarabine to the anthracycline is from 30:1 to 50:1.

6. The lipid nanoparticle composition of claim 1, wherein the effective mean particle size of the lipid nanoparticles is less than 200 nm.

7. The lipid nanoparticle composition of claim 1, wherein the components of the lipid nanoparticles comprise at least one phosphatidylcholine, a charged lipid stabilizer, and a conditioning agent of phospholipid membrane fluidity.

8. The lipid nanoparticle composition of claim 1, wherein the charged lipid stabilizer is at least one selected from the group consisting of methoxypolyethylene glycol-distearylphosphatidylethanolamine and phosphatidylglycerol.

9. The lipid nanoparticle composition of claim 8, wherein the phosphatidylglycerol is selected from any one of the following or a mixture of several of the following: dimyristoylphosphatidylglycerol, dipalmitoylphosphatidylglycerol, dioleoylphosphatidylglycerol, and distearylphosphatidylglycerol.

10. The lipid nanoparticle composition of claim 7, wherein the phosphatidylcholine is any one selected from egg yolk phosphatidylcholine, hydrogenated soybean phosphatidylcholine, distearoylphosphatidylcholine, dipalmitoylphosphatidylcholine, dioleylphosphatidylcholine, and dimyristoylphosphatidylcholine.

11. The lipid nanoparticle composition of claim 7, wherein the conditioning agent of phospholipid membrane fluidity is cholesterol.

12. A pharmaceutical composition comprising the lipid nanoparticle composition of claim 1 and a pharmaceutically acceptable carrier.

13. A pharmaceutical composition comprising the lipid nanoparticle composition of claim 1 and a pharmaceutically acceptable carrier, and the pharmaceutical composition comprises 1%-7% by weight of cytarabine, 0.1%-3% by weight of the anthracycline, 5%-20% by weight of hydrogenated soybean phosphatidylcholine or distearoylphosphatidylcholine, 0.5%-5% by weight of methoxypolyethylene glycol-distearylphosphatidylethanolamine, 0.5%-5% by weight of cholesterol, and 70%-90% by weight of sucrose.

14. The pharmaceutical composition of claim 13, comprising 2%-5% by weight of cytarabine, 0.1%-1.5% by weight of the anthracycline, 6%-12% by weight of hydrogenated soybean phosphatidylcholine or distearoylphosphatidylcholine, 1%-3% by weight of methoxypolyethylene glycol-distearylphosphatidylethanolamine, 1%-3% by weight of cholesterol, and 75%-85% by weight of sucrose.

15. A pharmaceutical composition comprising the lipid nanoparticle composition of claim 1 and a pharmaceutically acceptable carrier, and the pharmaceutical composition comprises 1%-7% by weight of cytarabine, 0.1%-3% by weight of the anthracycline, 5%-20% by weight of hydrogenated soybean phosphatidylcholine or distearoylphosphatidylcholine, 0.5%-10% by weight of distearylphosphatidylglycerol, 0.5%-5% by weight of cholesterol, and 65%-90% by weight of sucrose.

16. The pharmaceutical composition of claim 15 comprising 2%-5% by weight of cytarabine, 0.1%-1.5% by weight of the anthracycline, 12%-18% by weight of hydrogenated soybean phosphatidylcholine or distearoylphosphatidylcholine, 2%-5% by weight of distearylphosphatidylglycerol, 0.5%-2% by weight of cholesterol,. and 70%-80% by weight of sucrose.

17. The pharmaceutical composition of claim 12, wherein the lipid nanoparticles are in liquid form or lyophilized form.

18. A method of treating a hematoproliferative disorder, comprising administrating an effective amount of the lipid nanoparticle composition of claim to a subject in need thereof; wherein
the hematoproliferative disorder is at least one of leukemia, malignant lymphoma, or multiple myeloma.

19. (canceled)

20. The pharmaceutical composition of claim 13, wherein the lipid nanoparticles are in liquid form or lyophilized form.

21. The pharmaceutical composition of claim 15, wherein the lipid nanoparticles are in liquid form or lyophilized form.

22. A method of treating a hematoproliferative disorder, comprising administrating an effective amount of the pharmaceutical composition of claim 12 to a subject in need thereof; wherein
the hematoproliferative disorder is at least one of leukemia, malignant lymphoma, or multiple myeloma.

23. A method of treating a hematoproliferative disorder, comprising administrating an effective amount of the pharmaceutical composition of claim 13 to a subject in need thereof; wherein
the hematoproliferative disorder is at least one of leukemia, malignant lymphoma, or multiple myeloma.

24. A method of treating a hematoproliferative disorder, comprising administrating an effective amount of the pharmaceutical composition of claim 15 to a subject in need thereof; wherein
the hematoproliferative disorder is at least one of leukemia, malignant lymphoma, or multiple myeloma.
